SEQUENCE LISTING (4 sequences)

SEQ ID NO: 1 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 2 - protein, 261 aa, Bacillus megaterium
MYTDLKDKVVVVTGGSKGLGRAMAVRFGQEQSKVVVNYRSNEEEALEVKKEIEQAGGQAIIVRGDVTKEEDVVNLVETAVKEFGTLDVMINNAGVENPVPSHELSLENWNQVIDTNLTGAFLGSREAIKYFVENDIKGNVINMSSVHEMIPWPLFVHYAASKGGMKLMTETLALEYAPKGIRVNNIGPGAIDTPINAEKFADPEQRADVESMIPMGYIGNPEEIASVAAFLASSQASYVTGITLFADGGMTKYPSFQAGRG

SEQ ID NO: 3 - DNA, 807 bp, Saccharomyces cerevisiae
atgagccagggtcgtaaagcagcagaacgtctggcaaaaaaaaccgttctgattaccggtgcaagcgcaggtattggtaaagcaaccgcactggaatatctggaagcaagcaatggcgatatgaaactgattctggcagcacgtcgtctggaaaaactggaagaactgaaaaaaaccatcgatcaggaatttccgaacgcaaaagttcatgttgcacagctggatattacccaggcagaaaaaatcaaaccgtttatcgaaaatctgccgcaggaattcaaagatatcgatattctggtgaataatgcaggtaaagcactgggtagcgatcgtgttggtcagattgcaaccgaagatatccaggatgtgtttgataccaatgtgaccgcactgattaatattacacaggccgttctgccgatttttcaggcaaaaaacagcggtgatattgtgaatctgggtagcattgcaggtcgtgatgcatatccgaccggtagcatttattgtgcaagcaaatttgcagttggtgcatttaccgacagtctgcgcaaagaactgattaataccaaaatccgcgttattctgattgcaccgggtctggttgaaaccgaattcagcctggttcgttatcgtggtaatgaagaacaggccaaaaacgtgtataaagataccacaccgctgatggcagatgatgttgccgatctgattgtttatgcaaccagccgtaaacagaataccgttattgccgataccctgatttttccgaccaatcaggcatctccgcatcatatttttcgtggttaataa

SEQ ID NO: 4 - DNA, 789 bp, Bacillus megaterium
atgtataccgacctgaaagataaagttgttgttgtgaccggtggtagcaaaggtctgggtcgtgcaatggcagttcgttttggtcaggaacagagcaaagttgttgtgaattatcgcagcaatgaagaagaagccctggaagtcaaaaaagaaattgaacaggcaggcggtcaggcaattattgttcgtggtgacgtgaccaaagaagaggacgttgttaatctggttgaaaccgcagttaaagaatttggcaccctggatgtgatgattaataatgccggtgttgaaaatccggttccgagccatgaactgagcctggaaaattggaatcaggtgattgataccaatctgaccggtgcatttctgggtagccgtgaagccattaaatattttgtggaaaatgatattaaaggcaatgtgatcaatatgagcagcgttcatgaaatgattccgtggcctctgtttgttcattatgcagcaagcaaaggtggtatgaaactgatgaccgaaaccctggcactggaatatgcaccgaaaggtattcgtgtgaataatattggtccgggtgcaattgataccccgatcaatgcagaaaaatttgcagatccggaacagcgtgcagatgttgaaagcatgattccgatgggttatattggcaatccggaagaaattgcaagcgttgcagcatttctggcaagcagccaggcaagctatgttaccggtattaccctgtttgcagatggtggtatgaccaaatatccgagctttcaggcaggtcgtggttaataa

(19) United States
(12) Patent Application Publication
(10) Pub. No.: US 2014/0147896 A1
(43) Pub. Date: May 29, 2014

(54) ENZYME FOR THE PRODUCTION OF OPTICALLY PURE 3-QUINUCLIDINOL

(71) Applicant: CADILA HEALTHCARE LIMITED, Ahmedabad (IN)

(72) Inventors: Rupal Joshi, Gujarat (IN); Anita Nair, Gujarat (IN); Anita Ramrakhiani, Gujarat (IN); Sanjeev Kumar Mendiratta, Gujarat (IN); Umang Trivedi, Gujarat (IN)

(73) Assignee: CADILA HEALTHCARE LIMITED, Ahmedabad (IN)

(21) Appl. No.: 13/809,306

(22) PCT Filed: Jul. 14, 2011
(86) PCT No.: PCT/IN2011/000469

§ 371 Date: May 20, 2013

(30) Foreign Application Priority Data
Jul. 14, 2010 (IN) 2028/MUM/2010

(51) Int. Cl. C12P 17/18; C12N 9/04; C12N 9/02

(52) U.S. Cl. CPC C12P 17/182; C12N 9/0008; C12N 9/0006
USPC 435/119; 435/320.1

(57) ABSTRACT

The present invention provides a process for production of optically pure quinuclidinol of formula-(I) by reduction of quinuclidinone of formula-(II) in presence of suitable oxidoreductase enzyme derived from Saccharomyces species. Formula (II, I) Moreover, the present enzyme works in presence of cofactor NADP where the cofactor is regenerated by suitable system. The present invention also provides a recombinant vector containing genes co expressing suitable polypeptides having oxido-reductase activity and polypeptide having capacity to regenerate the co-factor. The said vector is transformed in suitable host cell.

### FIELD OF THE INVENTION

The invention relates to newly identified polynucleotide sequences that encode polypeptides having oxidoreductase enzymatic activity. The invention provides a process for the preparation of alcohols where the polypeptides of the present invention can convert suitable ketones to the corresponding alcohols stereoselectively. In particular, said polynucleotide sequences is cloned in a vector which enantio-selectively reduces the ketone of formula (II) to the corresponding-alcohol of formula (I) in optically pure form.

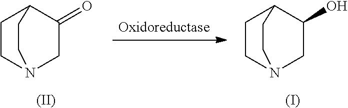

Further the present invention also discloses cofactor regeneration system through substrate based or enzyme based system to regenerate the cofactor during the reaction.

### BACKGROUND OF THE INVENTION

(R)-3-quinuclidinol is an optically active intermediate which is used in preparation of several drugs like Solifenacin, Polonosetron, Talsaclidine, Revatropate, for preparation of cholinergic receptor ligands and anesthetics and in addition, act as a key precursor for several pharmaceuticals used in the treatment of Alzheimer's disease and asthma.

There are few processes reported in prior art for the production of 3-quinuclidinol from a quinuclidinone but many of the process does not provides satisfactory yield and enantiomeric excess of the desired intermediate.

The stereospecific reduction of carbonyl groups can be used to produce chiral alcohols. Several biochemical and chemical approaches have been employed in the synthesis of enantiomerically pure alcohols. These approaches include stereospecific chemical reduction of ketones, enzymatic reduction of ketones, enzymatic hydrolysis of racemic esters, and enzymatic esterification of racemic alcohols.

J. Am. Chem. Soc. 74 (1952) 2215-2218 discloses a chemical resolution process of racemic quiniclidinol where a diastereomeric salt of (R)-3-quinuclidinol with (S)-camphorsulfonic acid in a solution of i-PrOH/acetone was prepared. However the diastereomeric salt was obtained in low overall yield of 6% after two recrystallization steps.

Jpn. Kokai Tokkyo Koho 2003, 155293, 2003 discloses another synthetic resolution process which involved catalytic hydrogenation of quinuclidinone over a rhodium complex bearing chiral C2-symmetric diphosphinite dubbed as d-CandyPhos. The optical purity of the product, (R)-3-quinuclidinol was 37% ee

The drawbacks associated with the classical methods to synthesize chiral alcohols from corresponding ketone compounds by conventional chemistry procedures is that it involves multiple steps like reduction, chiral resolution and recycling of unwanted isomer. In addition these chemical methods are very difficult to execute and do not always provide sufficient yield.

Furthermore, the above described chemical steps involve usage of organic solvents and complex procedure. Moreover, one of the major drawback of the chemical procedure is that during resolution step, theoretically only 50% of the total material can be isolated from the racemic mixture as a pure enantiomer. Thus wastage of 50% unwanted material makes the procedure costly and has an adverse effect on the environment. Also recycling of the wrong isomer requires extra unit operations and cost.

With the advent of biotechnology several enzymatic processes have been developed to obtain enantiomerically pure compound. Enzymes have unique stereo selective property therefore enzymatic reduction has the advantage of having only one enantiomer with good chiral purity.

Microbial enzymes have been used for the synthesis of chiral alcohols at laboratory, pilot, and production scale (C. J. Sih and C.-S. Chen, 1984, Angew Chem. Int. Ed. Engl. 23:570-578; O. P. Ward and C. S. Young, 1990, Enzyme Microb. Technol. 12:482-493).

Several processes for synthesizing optically active 3-quinuclidinol derivatives are known as described in e.g. Acta. Pharm. Suec., 16, 281-3 (1979), U.S. Pat. No. 3,997,543, Life sci. 21, 1293-1302 (1977) and U.S. Pat. No. 5,215,918 but none of the process provides satisfactory yield and enantiomeric excess of the desired intermediate.

U.S. Pat. No. 5,215,918 discloses enantiomeric enrichment of 3-quinuclidinol, by using subtilisin protease derived from Bacillus. However, the process does not provide satisfactory yield and enantiomeric excess of the desired intermediate

Acta Pharm. Suec. 1979, 16(4), 281-3, discloses a process which comprises hydrolyzing an acetylated racemic substrate, 3-quinuclidinol after resolving with tartaric acid. A drawback associated with the chemical resolution is that only 50:50 resolutions take place. Therefore, around 50% of the wrong enantiomer has to be thrown away. Large amounts of solvents and organic compounds are needed for a chemical reaction, ultimately contributing to cost and the environmental hazards. In contrast, the enzymatic reactions are quick, single pot, and operate under mild conditions, giving good yield and purity.

The resolution of racemic mixtures of 3-quinuclidinol derivatives to give optically active (R)-3-Quinuclidinol has been reported by Rehavi et al., in 21 Life Sciences 1293 (1977). This prior art discloses the enzyme-catalyzed hydrolysis of (R)-3-quinuclidinol butyrate by butyrylcholine esterase obtained from horse serum. However, the enantioselective hydrolysis of the (R)-ester to obtain R-alcohol was very slow (about 10 hours), resulting in low recovery and relatively low enantiomeric enrichment of the R-enantiomer.

U.S. Pat. No. 5,888,804 discloses processes for producing an optically active quinuclidinol from a quinuclidinone by an asymmetric reduction using a microorganism and enzyme. The microorganisms disclosed as being capable of producing an (R)-3-quinuclidinol from a 3-quinuclidinone included those selected from the genus Nakazawaea, the genus Candida and the genus Proteus. The microorganisms capable of producing an (S)-3-quinuclidinol from a 3-quinuclidinone are selected from the genus Arthrobacter, the genus Pseudomonas and the genus Rhodosporidium.

However, the substrate concentration used in this process is not industrially viable and the product suffers from a low optical purity.

JP-A Hei 10-210997, discloses the use of Esterases derived from the genus Aspergillus or the genus Pseudomonas for the production of optically active 3-quinuclidinol.

JP-A Hei 10-136995 disclosed cells of microorganisms belonging to the genus Aspergillus, the genus Rhizopus, the genus Candida, and the genus Pseudomonas and enzymes derived there from for the production of optically active 3-quinuclidinol.

EP0945518 discloses synthesis of optically active 3-quinuclidinol derivatives, by using enzymes selected from the genus Aspergillus, Rhizopus, Candida or Pseudomonas. However the yield of the product in the respective reaction is only around 25%.

JP-A Hei 10-243795, JP-A Hei 11-196890, JP-A 2000-245495, Abstract (2001). The Japan Agricultural Chemical Society, pp. 3713Y7a9 disclosed a process for the production of optically active 3-quinuclidinol from 3-quinuclidinone, which comprises asymmetric reduction using suitable microorganisms or enzymes.

Japan. Kokai Tokkyo Koho 99, 196890 (1999) discloses the production of (R)-3-Quinuclidinol, in 4 days duration with 65% yield, comprising a whole cell biotransformation route.

However, the product obtained by these production methods has either low chiral purity and/or poor recovery. In addition, these production methods have complicated and multiple synthesis steps.

In addition, the prior art process as mentioned in “Stereoselective synthesis of (R)-3-quinuclidinol through a symmetric reduction of 3-quinuclidinone with Rhodotorula rubra”, in Applied Microbiol Biotechnol; 83, 617-626, 2009, operates at significantly low volumetric capacity and further requires externally added cofactors, which play a crucial role in selectivity and efficiency of oxidoreductase activities to produce chiral compounds. These naturally available co-factors like NADPH and NADH generally act as electron carrier facilitating redox biotransformations and are consumed at stoichiometric ratios. However these cofactors are more expensive when added externally than the enzymes and increase the cost of final product.

Therefore, the present invention further provides a co-factor regeneration system which is selected from substrate or enzyme based regeneration system.

U.S. Pat. No. 7,645,599 describes the production of optically active 3-quinuclidinol by co-expressing tropinone reductase-1 with glucose dehydrogenase where the tropinone reductase-I catalyze the reduction of optically active 3-quinuclidinol and glucose dehydrogenase regenerates reduced co-factor NAD(P)H from NADP. However this process was time consuming.

Hence, there has been a long felt need in the art for a simple, efficient and inexpensive method of making optically pure (R)-3-quinuclidinol. The inventors of the present invention surprisingly have found novel enzymes from microorganisms which provides high yield of optically pure (R)-3-quinuclidinol and also improve the production efficiency and further the present process can be carried out at high volumetric capacity in significantly lesser reaction time which is equal to or better than those of the existing processes. Further the inventors have made intensive studies to construct a vector to co-express the above described polynucleotide sequences having oxidoreductase activity and co-factor regenerating activity in a single expression system and thereby the present invention removes the need of external addition of co-factor during the reaction and to provide a simple, cost effective and industrial viable process for the production of optically active (R)-3-quinuclidinol. Further, this process was found to be highly scalable and cost effective at an industrial scale.

### OBJECTS OF THE INVENTION

It is therefore, an object of the present invention to provide process for preparing optically active (R)-3-quinuclidinol of formula (I), from the corresponding quinuclidinone of formula (II) using enzymatic asymmetric stereoselective reduction.

In one embodiment the invention provides enantioselective reduction of a ketone with oxidoreductases of the present invention to corresponding alcohols and provides optically pure form of product.

In an embodiment of the present invention provides oxidoreductase enzyme having a specific enantioselective function to catalyze the production of optically pure (R)-3-quinuclidinol.

In an embodiment of the present invention is provided the nucleotide sequence encoding the polypeptide as described in this invention.

In another embodiment of the present invention is provided a nucleotide sequence encoding the polypeptide having at least one amino acid substitution, insertion, deletion and addition thereof.

In yet another embodiment of the present invention provides an oxidoreductase enzyme and nucleotide and polypeptide sequence thereof derived from Saccharomyces species.

In yet another embodiment the oxidoreductase enzyme is prepared by recombinant technology.

In a further embodiment of the present invention provides an expression vector comprising gene encoding the desired polypeptide having oxidoreductase enzymatic activity.

In yet another embodiment of the present invention provides a polycistronic vector comprising the polynucleotide sequence encoding polypeptide having oxidoreductase activity and further the polynucleotide sequence encoding polypeptide having potential to generate co-factor from oxidized NADP.

Accordingly, in embodiment it is an object of the invention to provide a method for co-expressing oxidoreductase enzyme and polypeptide having potential to generate co-factor.

In yet another embodiment of the present invention provides co-factor regenerative systems selected from substrate coupled or enzyme coupled systems.

A further embodiment of the present invention provides a process for the production of 3-quinuclidinol of formula-(I) by reduction of quinuclidinone of formula-(II) in the presence of oxidoreductase enzyme derived from Saccharomyces species.

In a still further embodiment of the present invention provides a process of production of 3-quinuclidinol of formula-(I) by reduction of quinuclidinone of formula-(II) using whole cell biocatalysis.

In yet another embodiment of the present invention provides for over-expression of the desired polypeptide having the desired oxidoreductase enzymatic activity in E. coli transformed cells.

### SUMMARY OF INVENTION

The present invention provides a process for production of optically pure 3-quinuclidinol of formula-(I) by reduction of quinuclidinone of formula-(II) in the presence of suitable oxidoreductase enzyme derived from Saccharomyces species.

In embodiment the present invention provides a process for preparing the 3-quinuclidinol of formula (I) which comprises
a. reacting the quinuclidinone of formula (II) with an oxidoreductase enzyme of sequence ID no. 1 or its suitable variants in the presence of suitable cofactor in suitable solvent;
b. isolating the 3-quinuclidinol.

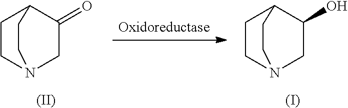

Moreover, the present enzyme works in presence of cofactor NADP where the cofactor is regenerated by substrate coupled or enzyme coupled system. The present invention also provides a recombinant vector containing genes coding for suitable polypeptides which show oxido-reductase activity and also codes polypeptide having capacity to regenerate the co-factor. The said vector is transformed in suitable host cell.

### BRIEF DESCRIPTION OF DRAWING

FIG. 1 depicts the nucleotide (Seq ID. 3) and polypeptide sequences (Seq ID. 1) of oxidoreductase enzyme.

FIG. 2 depicts the nucleotide (Seq ID. 4) and polypeptide sequences (Seq ID. 2) of Glucose Dehydrogenase enzyme.

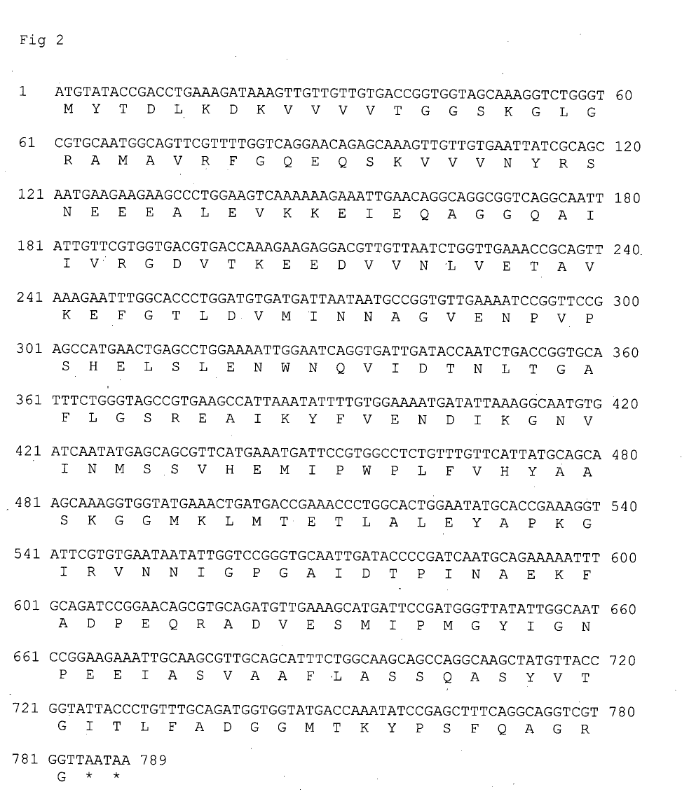

FIG. 3 depicts the map of recombinant plasmid pET27b2.0.1.

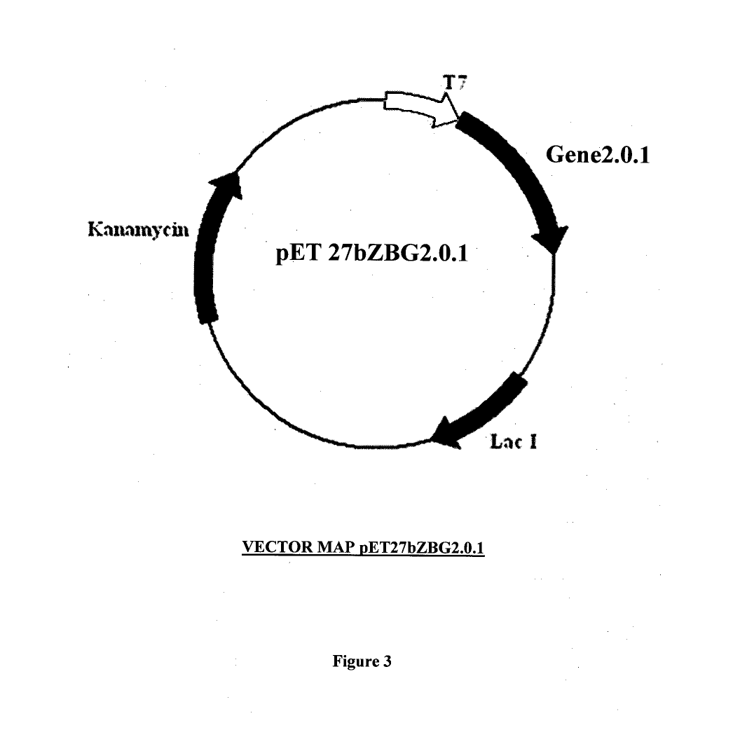

FIG. 4 depicts the map of recombinant plasmid pZRC2G-1ZBG2.0.1c1

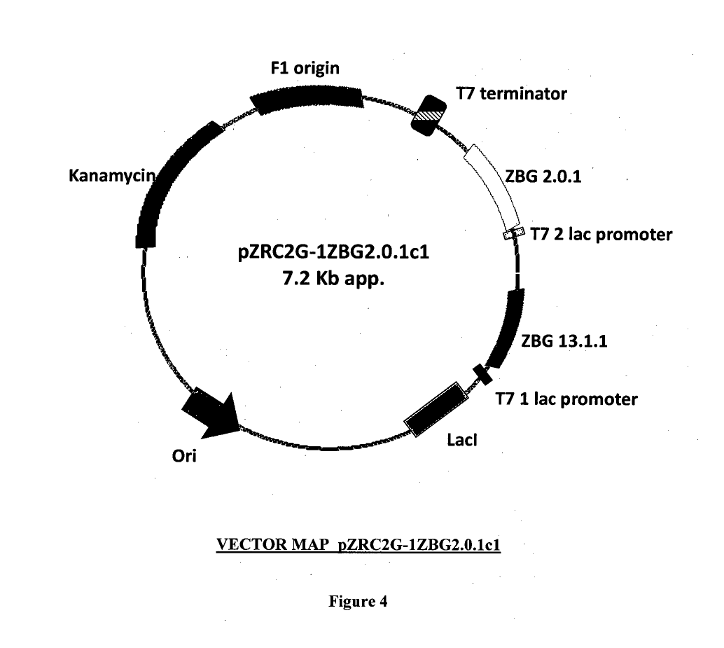

FIG. 5 depicts the map of recombinant plasmid pZRC2G-1ZBG2.0.1c2

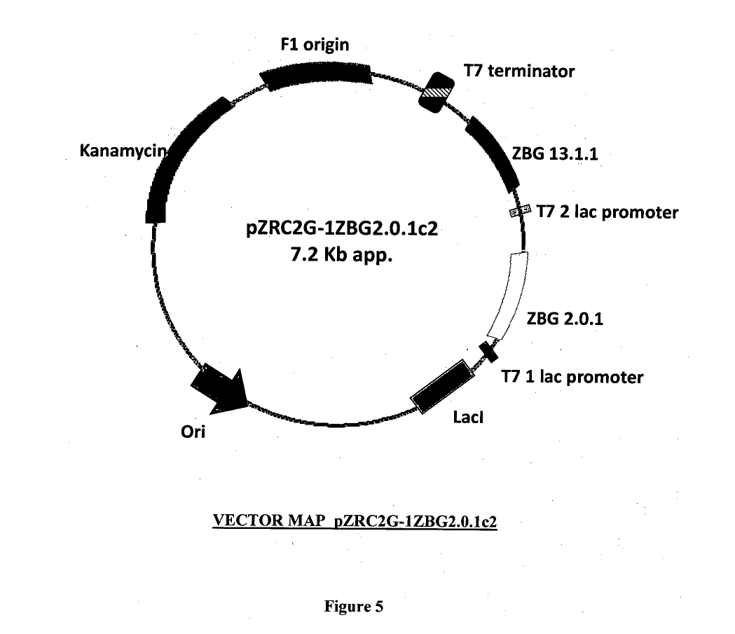

### DESCRIPTION OF THE EMBODIMENTS

### Definitions

The term “variants” as described herein refers to polypeptide derived from nucleotide sequence of sequence id no. 1, by addition, deletion, substitution and insertion of at least one nucleotide.

The term “expression construct” as used herein containing a nucleotide sequence of interest to express and control element.

The term as used herein “monocistronic expression construct” means that a gene expressed in a single expression construct.

The term as used herein “polycistronic expression construct” means that two or more gene expressed in a single expression construct.

The term as used herein “enzyme coupled co-factor regeneration system” means the expression of suitable polypeptide in vector having potential to regenerate cofactor from oxidized NADP during the reaction.

The term as used herein “substrate coupled co-factor regeneration system” means the use of a suitable substrate H⁺ donor having potential to regenerate cofactor from oxidized NADP during the reaction.

The term as used herein “pZRC2G-1ZBG2.0.1c1” means the expression vector comprises
a. At least one region that control the replication and maintenance of said vector in the host cell
b. first region comprising in the direction of transcription from the first promoter, a first promoter operably linked to the nucleotide sequence set forth in sequence ID no 1 encoding oxidoreductase enzyme
c. Second region comprising in the direction of transcription from the first promoter, a second promoter operably linked to the nucleotide sequence set forth in sequence ID no 2 encoding polypeptide having potential to regenerate co-factor.

Wherein the first and second region is contiguous.

The term as used herein “pZRC2G-1ZBG2.0.1c2” means the expression vector comprises
a. At least one region that control the replication and maintenance of said vector in the host cell
b. first region comprising in the direction of transcription from the first promoter, a first promoter operably linked to the nucleotide sequence set forth in sequence ID no 2 encoding polypeptide having potential to regenerate co-factor
c. Second region comprising in the direction of transcription from the first promoter, a second promoter operably linked to the nucleotide sequence setforth in sequence ID no 1 encoding oxidoreductase enzyme

wherein the first and second region is contiguous.

The term used herein “whole cell” means a recombinant E. coli deposited under Budapest treaty, having accession number MTCC 5621

The present invention discloses enzymatic reduction of 3-quinuclidinone to produce optically pure 3-quinuclidinol, which is a useful intermediate for preparation of active pharmaceuticals. Thus the present invention discloses novel polypeptide and its variants which have enzymatic activity to catalyze the reduction of quinuclidinone of formula (II), to produce optically pure (R)-3-quinuclidinol of formula (I).

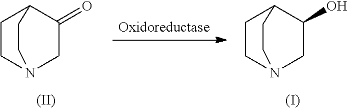

In one embodiment of the present invention the polypeptide having desired enzymatic activity and variants thereof can be isolated from suitable bacteria, yeast or fungi. In a preferred embodiment the present polypeptide is isolated from the Saccharomyces species. In a further preferred embodiment the present polypeptide is isolated from Saccharomyces cerevisiae. Thereafter the polypeptide of interest having the desired enzymatic activity is purified by techniques known in the art. In another embodiment, the polypeptide and its variants can be synthesized chemically according to known processes such as those described in “Total synthesis of a gene. Khorana HG; Science 1979 Feb. 16 203(4380:614-25”

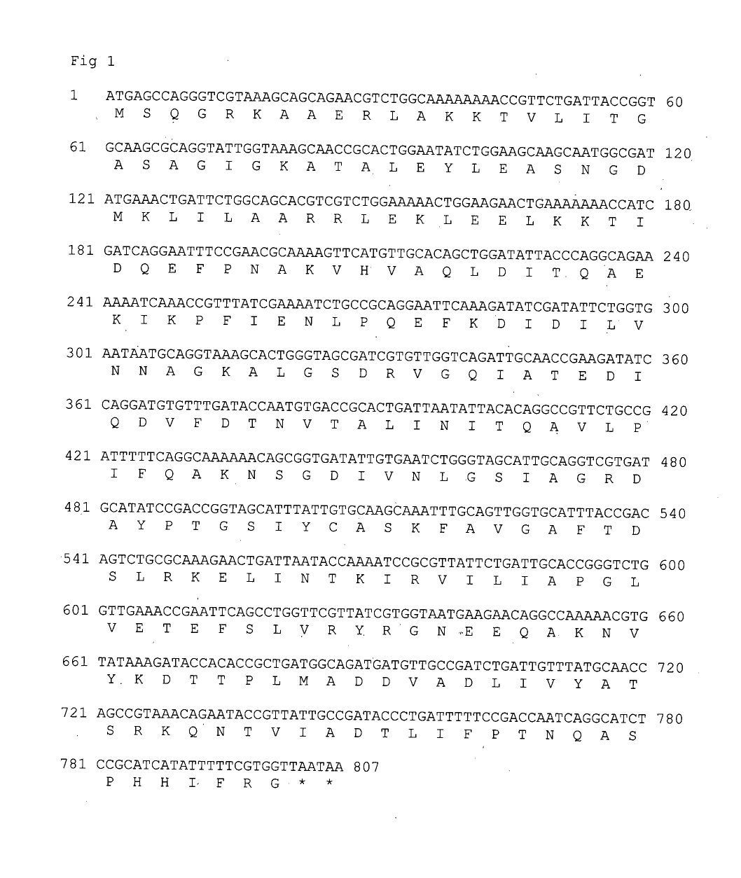

In one embodiment the polynucleotide and its variants encode the polypeptide of the present invention having oxidoreductase enzymatic activity. The polynucleotide has the sequence id 1 as given in FIG. 1. In a preferred embodiment the polynucleotide and its variants according to the sequence id no. 1 have short chain dehydrogenase enzymatic activity. In a further preferred embodiment, the polynucleotide and its variants according to the sequence id no. 1 comprises a short chain alcohol dehydrogenase which has a ketoreductase enzymatic activity. In the more preferred embodiment the polynucleotide and its variants according to the sequence id no. 1 comprises short chain alcohol dehydrogenase enzymatic activity.

The term “variants” as described herein refers to polypeptide derived from nucleotide sequence of sequence id no. 1, by addition, deletion, substitution and insertion of at least one nucleotide.

In the present invention, it is possible to use not only the native enzyme but also a mutant enzyme comprising an amino acid sequence in which one or more amino acid residues have been substituted, deleted, and/or inserted as compared with the original amino acid sequence, so long as the mutant enzyme has the activity of producing (R)-3-quinuclidinol by reducing 3-quinuclidinone.

In one embodiment, the polypeptide and its variants, which have the desired enzymatic activity, are encoded by the nucleotide sequence of seq. id no. 1. A preferred embodiment of the present invention comprises polynucleotide sequence encoding the polypeptide of the present invention having enzymatic activity which is at least 50% identical to those nucleotide sequences disclosed in sequence id no 1.

In one aspect the genes of desired oxidoreductase enzyme is derived from Saccharomyces species more specifically from Saccharomyces cerevisiae. In another embodiment genes of desired oxidoreductase enzyme activity can be isolated from various organisms by hybridizing the nucleotide sequence of sequence i.d. no. 1 or a partial sequence thereof, obtained from the cDNA sequence as a probe to DNAs prepared from other organisms under stringent conditions. The polynucleotides capable of hybridizing under stringent condition refers to a polynucleotide capable of hybridizing to a DNA comprising a nucleotide sequence corresponding to the amino acid sequence of SEQ ID NO: 1 as the probe, for example, by using ECL™ direct nucleic acid labeling and detection system (Amersham Pharmacia Biotech) under the condition as described in the manufacturer's instruction (wash at 42° C. with a primary wash buffer containing 0.5 times SSC).

Furthermore, based on the above-mentioned nucleotide sequence information, PCR primers can be designed from regions exhibiting high homology. The gene encoding short chain alcohol dehydrogenase can be isolated from various organisms by PCR using such primers and chromosomal DNA or cDNA as a template.

The polynucleotides or its variants of desired enzymatic activity are cloned into suitable vectors which can be selected from plasmid vector, a phage vector, a cosmid vector and shuttle vector may be used that can exchange a gene between host strains. Such a vector typically includes a control element, such as a lacUV5 promoter, a trp promoter, a trc promoter, a tac promoter, a lpp promoter, a tufB promoter, a recA promoter, or a pL promoter, and is preferably employed as an expression vector including an expression unit operatively linked to the polynucleotide of the present invention. In a preferred embodiment the polynucleotide of sequence id no. 1 and its variants is cloned in a cloning vector construct pET11, according to general techniques described in Sambrook et al, Molecular cloning, Cold Spring Harbor Laboratories (2001). The constructed vector is now onwards referred to as pET11aZBG2.0.1. The term “control element” as used herein refers to a functional promoter and a nucleotide sequence having any associated transcription element (e.g., enhancer, CCAAT box, TATA box, SPI site).

The polynucleotide of the present invention is linked with controlling elements, such as a promoter and an enhancer, which controls the expression of the polynucleotide in such a manner that the controlling elements can operate to express the gene. It is well known to those skilled in the art that the types of control elements may vary depending on the host cell.

The above described vector further contains a genes of enzymes which regenerate the co-factor such as NAD, NADP, NADH, NADPH

In an embodiment the present process provides a vector construct comprising monocistronic expression construct of nucleotide sequence encoding the polypeptide having desired oxidoreductase enzymatic activity. Alternatively vector construct comprising monocistronic expression construct of nucleotide sequence encoding the polypeptide have the potential to generate co-factor from oxidized NADP during the reaction.

According to such embodiment the oxidoreductase polypeptide encoded by sequence id no. 1 is coupled with the cofactor selected from NAD(P)H/NAD(P) to produce the optically pure 3-quinuclidinol of formula-(I) by reduction of the quinuclidinone of formula-(II) wherein the cofactor is either added externally in reaction medium or obtained by enzyme/substrate coupled regeneration system.

In an embodiment the present process provides a vector construct comprising polycistronic expression construct of nucleotide sequences encoding the polypeptide having desired oxidoreductase enzymatic activity and the polypeptide having potential to generate co-factor from oxidized NADP during the reaction.

According to such embodiment the oxidoreductase polypeptide encoded by sequence id no. 1 is coupled with the cofactor selected from NAD(P)H/NAD(P) to produce the optically pure 3-quinuclidinol of formula-(I) by reduction of the quinuclidinone of formula-(II) wherein the cofactor is co expressed with nucleotide sequence encoding polypeptide having oxidoreductase activity in the same vector.

In an embodiment the vector is having potential to co-express oxidoreductase polypeptide encoded by sequence id no. 1 with polypeptide having potential to generate co-factor from oxidized NADP during the reaction comprises
a. At least one region that control the replication and maintenance of said vector in the host cell;
b. first promoter operably linked to the nucleotide sequence setforth in sequence ID no 1 (ZBG2.0.1) encoding the oxidoreductase enzyme;
c. Second promoter operably linked to the nucleotide sequence setforth in sequence ID no 2 (ZBG13.1.1) encoding polypeptide having potential to regenerate co-factor;
d. Suitable antibiotic marker

In an embodiment the gene positions are changeable and therefore position of sequence ID no 2 (ZBG13.1.1) can be replaced by sequence ID no 1 (ZBG2.0.1) or position of sequence ID no 1 (ZBG2.0.1) may be replaced by sequence ID no 2 (ZBG13.1.1).

According to the present invention monocistronic or polycistronic vector containing polynucleotides or its variants having desired oxidoreductase enzymatic activity is transfected in to the host cells using a calcium chloride method as known in the art. The host cell may be selected from bacteria, yeast, molds, plant cells, and animal cells. In a preferred embodiment the host cell is a bacteria such as Escherichia coli. In such embodiment the above mentioned desired polynucleotides is over-expressed in E. coli

According to preferred embodiment the invention provide a process for the production of the compound of formula (I) which comprises
a) Dissolution of the compound of formula (II) in suitable solvent;
b) Reacting the compound of formula (II) with suitable oxidoreductase enzyme in the presence of suitable cofactor;
c) Optionally maintain the pH during the reaction
d) Isolation of the compound of formula (I)

The oxidoreductase enzymes suitable for the reaction shares at least 50% homology/identity with the sequence ID no. 1 or its variants.

In one such embodiment the cofactor is added externally in reaction medium. In an alternate embodiment the co factor is obtained by enzyme coupled regeneration system. The enzyme which is used in enzyme coupled regeneration system is selected from glucose dehydrogenase, formate dehydrogenase, malate dehydrogenase, glucose-6-phosphate dehydrogenase, phosphite dehydrogenase. In one preferred embodiment the enzyme is glucose dehydrogenase. In one such embodiment oxidoreductase enzyme is expressed in monocistronic vector. In another embodiment oxidoreductase enzyme is co-expressed with glucose dehydrogenase in polycistronic vector in a single expression system. In such a preferred embodiment, the expression system is bacteria such as Escherichia coli.

In another embodiment, oxidoreductase polypeptide encoded by sequence id no. 1 is coupled with the cofactor selected from NAD(P)H/NAD(P) to produce the optically pure 3-quinuclidinol of formula-(I) by reduction of the quinuclidinone of formula-(II) wherein the cofactor is regenerated through substrate coupled regeneration system.

The substrate coupled regeneration system comprises co-substrate selected from ethanol, 2-propanol, 4-methyl-2-pentanol, 2-heptanol, 2-pentanol, 2-hexanol. In preferred embodiment the co-substrate used in substrate coupled regeneration system is 2-propanol.

Moreover, the substrate coupled regeneration system requires the action of at least one enzyme. In preferred embodiment the substrate coupled regeneration system requires the action of enzyme comprises the polypeptide as set forth in sequence id no 1 or variants thereof. According to preferred embodiment of the process sequence id no 1 or variants is expressed in monocistronic vector.

According to preferred embodiment the reduced co-factor such as NAD(P)H is regenerated by dehydrogenation of the 2-propanol by the enzyme of sequence id no 1 to produce acetone. Furthermore the reduced co-factor couples with the said enzyme and reacts with substrate according to acid-base catalytic mechanism. Thus, in this process the reduced co-factor NAD(P)H is regenerated continuously by dehydrogenation of alcohol by the same oxidoreductase enzyme.

In one embodiment the optically pure chiral 3-quinuclidinol of formula (I) is prepared by reacting the quinuclidinone of formula (II) in suitable reaction condition with the cell-free extracts which comprises the desired polynucleotide or its variants according to sequence id no. 1. The cell free extract is obtained from the lysis of the host cell comprising the monocistronic vector containing the polynucleotide sequence encoding the oxidoreductase enzyme and its variants according to sequence id no. 1 and the required cofactor may be added externally. Alternatively, the cell free extract is obtained from the lysis of the host cell comprising the polycistronic vector containing the polynucleotide sequence encoding the oxidoreductase enzyme and its variants according to sequence id no. 1 and polypeptide in vector having potential to regenerate cofactor from oxidized NADP.

Optionally the cell free extract may be lyophilized or dried to remove water by the processes known in the art such as lyophilization or spray drying. The dry powder obtained from such processes comprises at least one oxidoreductase enzyme and its variants according to sequence id no. 1 which may be used to form optically pure chiral 3-quinuclidinol of formula (I) from quinuclidinone of formula (II).

In an embodiment the optically pure chiral 3-quinuclidinol of formula (I) is prepared by reacting the quinuclidinone of formula (II) in suitable reaction condition with the whole cells biocatalyst which comprises at least the desired polypeptide or its variants encoded by nucleotide sequence as set forth in sequence id no. 1 and the cofactor may be added externally during the reaction.

According to the preferred embodiment invention provides a process for the production of the compound of formula (I) which comprises
a) Dissolution of the compound of formula (II) in suitable solvent
b) Reacting the compound of formula (II) with suitable recombinant whole cell which comprises an expression vector which co-expresses the oxidoreductase enzyme and polypeptide having potential to regenerate co-factor, wherein the oxidoreductase enzyme of the sequence ID no. 1 and its variants.
c) Maintain the pH during the reaction
d) Isolation of the compound of formula (I)

The oxidoreductase enzyme suitable for the reaction as described above shares at least 50% homology/identity with the sequence ID no. 1 or its variants.

In such embodiment the whole cell is selected from recombinant E. coli having accession number MTCC 5621 which expresses the desired polypeptide or its variants encoded by nucleotide sequence as set forth in sequence id no. 1 and polypeptide having capacity to regenerates the reduced form of NAD(P)H.

In yet another embodiment the optically pure chiral 3-quinuclidinol of formula (I) is prepared by reacting the quinuclidinone of formula (II) in suitable reaction condition with the isolated and purified desired polypeptide encoded by polynucleotide as shown in sequence id no. 1 or its variants which shows at least 50% homology with the sequence id no. 1.

In preferred embodiment the optically pure chiral 3-quinuclidinol of formula (I) is prepared by reacting the quinuclidinone of formula (II) in suitable reaction condition with isolated and purified polypeptide encoded by polynucleotide of sequence id no. 1 or its variants which shows at least 50% homology with the sequence id no. 1 and further comprises the polypeptide having capacity to regenerates the reduced form of NAD(P)H.

In one general embodiment of the process according to the invention, the ketone of formula (II) is preferably used in an amount of from 0.1 to 30% W/V. In a preferred embodiment, the amount of ketone is 10% W/V. The process according to the invention is carried out in aqueous system. In such embodiment the aqueous portion of the reaction mixture in which the enzymatic reduction proceeds preferably contains a buffer. Such buffer is taken in the range of 50-200 mM is selected from sodium succinate, sodium citrate, phosphate buffer, Tris buffer. The pH is maintained from about 5 to 9 and the reaction temperature is maintained from about 15° C. to 50° C. In a preferred embodiment the pH value is 7-7.5 and the temperature ranges from 25° C. to 40° C.

Alternatively, the reaction can be carried out in an aqueous solvent in combination with organic solvents. Such aqueous solvents include buffers having buffer capacity at a neutral pH, are selected from phosphate buffer and Tris-HCl buffer. Organic solvents are selected from n-butanol, Iso propyl alcohol, ethyl acetate, butyl acetate, toluene, chloroform, n-hexane, ethanol, acetone, dimethyl sulfoxide, and acetonitrile etc. In another embodiment, the reaction is performed without buffer in presence of acid and alkali which maintain the pH change during the reaction within a desired range. Alternatively, the reaction can be carried out in a mixed solvent system consisting of water miscible solvents such as ethanol, acetone, dimethyl sulfoxide, and acetonitrile

The Polypeptide having desired enzymatic activity encoded by the nucleotide sequence as disclosed in sequence id no. 1 or its variants thereof is used in concentration of at least 5 mg/mL of lyophilized and water-resuspended crude lysate.

Furthermore, in such embodiment, optionally the NADP formed with the enzymatic reduction of NAD(P)H can again be converted to NAD(P)H with the oxidation of co substrate selected from Ethanol, 2-propanol, 4-methyl-2-pentanol, 2-heptanol, 2-pentanol, 2-hexanol. Moreover, the concentration of the cofactor NADP or NADPH respectively is selected from 0.001 mM to 100 mM.

In one preferred embodiment the reduction of the quniclidinone of formula (II) and the co-substrate is carried out by the same polypeptide encoded by polynucleotide of sequence id no. 1 or its variants.

In another embodiment the reduction of the quniclidinone of formula (II) and co-substrate is carried out by the polynucleotide of sequence id no. 1 in combination with the polypeptides selected from Glucose dehydrogenase, Formate dehydrogenase, Malate dehydrogenase, Glucose-6-Phosphate dehydrogenase, Phosphite dehydrogenase.

In such embodiment, the cofactor is regenerated by the oxidation of glucose used as co-substrate in the presence of Glucose dehydrogenase in suitable concentration such that its concentration is at least 0.1-10 times higher molar concentration than the keto substrate. In such embodiment the enzyme concentration is selected from at least 5 mg/mL of lyophilized and water-resuspended crude lysate.

In such embodiment the process of the invention is carried out closed reaction vessel made of glass or metal. For this purpose, the components are transferred individually into the reaction vessel and stirred or shaked for suitable hours preferably for 12 to 72 hours. In a preferred embodiment the reaction vessel is stirred or shaked for 3-12 hours. Thereafter the completion of the reduction, optically pure 3-quinuclidinol is recovered from suitable organic solvents after alkalifying with suitable bases, and thereafter analyzed by GC followed by chiral HPLC analysis.

According to the present invention, a process for the preparation of chiral 3-10 quinuclidinol of formula (I) from the quinuclidinone of formula (II) can be carried out by various processes including the use of recombinant host cell, cell free extract/crude lysate obtained from recombinant host cell, isolated desired enzyme which is isolated from cell free extract/crude lysate or from the suitable organism.

The invention is described in further details through the following examples which teach the skilled person to carry out the present invention. It will be appreciated that these examples are illustrative and the skilled person, following the teachings of these examples, replicate the teachings with suitable modifications, alterations etc. as may be necessary, and which are within the scope of a skilled person, for the entire scope which have been contemplated to be within the scope of the present invention.

### Example 1

### Construction of pET27bZBG2.0.1 for the Cloning and Expression Analysis of Oxidoreductase Enzyme

A codon optimized DNA sequence deduced from the polypeptide sequence as shown in sequence id no. 1 was cloned in a pET11a plasmid vector. The ligated DNA was further transformed into competent E. coli cells and the transformation mix was plated on Luria agar plates containing ampicillin. The positive clones were identified on the basis of their utilizing ampicillin resistance for growth on the above Petri plates and further restriction digestion of the plasmid DNA derived from them. Clones giving desired fragment lengths of digested plasmid DNA samples were selected as putative positive clones. One of such putative positive clones was submitted to nucleotide sequence analysis and was found to be having 100% homology with the sequence used for chemical synthesis. This clone was named pET11aZBG2.0.1. Plasmid DNA isolated from this clone was transformed into the E. coli expression host, BL21(DE3), and plated on ampicillin containing Luria Agar plates followed by incubation at 37° C. for overnight. Colonies picked from this plate were grown in Luria Broth containing ampicillin followed by induction with suitable concentration 2 mM of IPTG for expression analysis. Simultaneously the plasmid DNA isolated from the uninduced culture was further subjected to restriction digestion analysis using restriction enzymes SspI and Pvu I to confirm the correctness of the clone. IPTG induced cultures were lysed and clarified lysates obtained after centrifugation were subjected to SDS-PAGE analysis to confirm induced expression of polypeptide of correct size. Subcloning of the gene was done in pET27 b (+) having a kanamycin resistance gene instead of ampicillin. All other components of the vector were similar to pET11a. Briefly, the pET11aZBG2.0.1 plasmid DNA was digested with NdeI and BamHI to excise the gene from the vector. After digestion with these enzymes the DNA sequence was ligated with pET27b(+) plasmid vector pre-digested with NdeI and BamHI. The ligated DNA was further transformed into competent E. coli Top10F′ cells and the transformation mix was plated on Luria agar plates containing kanamycin. The positive clones were identified on the basis of their utilizing kanamycin resistance for growth on the above Petri plates and further restriction digestion of the plasmid DNA derived from them. The restriction enzymes, such as SspI, which is supposed to digest both the vector and the gene insert obtained from such clones was used for screening. One such clone giving desired fragment lengths of digested plasmid DNA samples was selected as a positive clone. This clone was named pET27bZBG2.0.1. Plasmid DNA isolated from this clone was transformed into the E. coli expression host, BL21 (DE3), and plated on kanamycin containing Luria Agar plates followed by incubation at 37° C. for overnight. Colonies picked from this plate were, grown in Luria Broth containing kanamycin followed by induction with suitable concentration 2 mM of IPTG for expression analysis. Simultaneously the plasmid DNA isolated from the uninduced cultures was further subjected to restriction digestion analysis using restriction enzymes SspI and Pvu I to confirm the correctness of the clone. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of this clone known as pET27bZBG2.0.1 was used for the preparation of glycerol stocks. This clone pET27bZBG2.0.1 was used as a source of enzymatic polypeptide of Seq ID no 1 for subsequent biocatalysis studies.

### Example 2

### Construction of pET27bZBG13.1.1 BL21 (DE3) for the Expression Analysis of Cofactor-Regenerating Enzyme (GDH)

A codon optimized DNA sequence encoding GDH deduced from the polypeptide sequence as shown in sequence id no. 2 was cloned in a pET11a plasmid vector. The ligated DNA was further transformed into competent E. coli cells and the transformation mix was plated on Luria agar plates containing ampicillin. The positive clones were identified on the basis of their utilizing ampicillin resistance for growth on the above Petri plates and further restriction digestion of the plasmid DNA derived from them. Clones giving desired fragment lengths of digested plasmid DNA samples were selected as putative positive clones. One of such putative positive clones was submitted to nucleotide sequence analysis and was found to be having 100% homology with the sequence used for chemical synthesis. This clone was named pET11aZBG13.1.1. Plasmid DNA isolated from this clone was transformed into the E. coli expression host, BL21(DE3), and plated on ampicillin containing Luria Agar plates followed by incubation at 37° C. for overnight. Colonies picked from this plate were grown in Luria Broth containing ampicillin followed by induction with suitable concentration 2 mM of IPTG for expression analysis. Simultaneously the plasmid DNA isolated from the uninduced cultures was further subjected to restriction digestion analysis using restriction enzymes Pvu II to confirm the correctness of the clone. IPTG induced cultures were lysed and clarified lysates obtained after centrifugation were subjected to SDS-PAGE analysis to confirm induced expression of polypeptide of correct size. Subcloning was done in pET27 b (+) having a kanamycin resistance gene instead of ampicillin. All other components of the vector were similar to pET11a. Briefly, the pET11aZBG13.1.1 plasmid DNA was digested with NdeI and BamHI to excise the gene from the vector. After digestion with these enzymes the DNA sequence was ligated with pET27b(+) plasmid vector pre-digested with NdeI and BamHI. The ligated DNA was further transformed into competent E. coli Top10F′ cells and the transformation mix was plated on Luria agar plates containing kanamycin. The positive clones were identified on the basis of their utilizing kanamycin resistance for growth on the above Petri plates and further restriction digestion of the plasmid DNA derived from them. The restriction enzymes, such as SspI, which is supposed to digest both the vector and the gene insert obtained from such clones was used for screening. One such clone giving desired fragment lengths of digested plasmid DNA samples was selected as a positive clone. This clone was named pET27b ZBG13.1.1. Plasmid DNA isolated from this clone was transformed into the E. coli expression host, BL21 (DE3), and plated on kanamycin containing Luria Agar plates followed by incubation at 37° C. for overnight. Colonies picked from this plate were grown in Luria Broth containing kanamycin followed by induction with suitable concentration 2 mM of IPTG for expression analysis. Simultaneously the plasmid DNA isolated from these uninduced cultures was further subjected to restriction digestion analysis using restriction enzymes SspI and Pvu I to confirm the correctness of the clone. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of this clone known as pET27bZBG13.1.1 BL 21(DE3) was used for the preparation of glycerol stocks. This clone pET27bZBG13.1.1 BL21 (DE3) was used as a source of enzymatic polypeptide of Seq ID no 2 for subsequent biocatalysis studies.

### Example 3

### Construction of Plasmid pZRC2G-1ZBG2.0.1c1 for Coexpression of Oxidoreductase and Cofactor Regenerating Enzyme

The plasmid pET27bZBG13.1.1 prepared according to example 2 containing a GDH gene deduced from the polypeptide sequence as shown in sequence id no. 2 was used for the co-expression of oxidoreductase derived from DNA sequence id no. 1 in single expression system. The expressions construct of the pET 11a ZBG 2.0.1 containing T7 promoter RBS and the ZBG 2.0.1 gene was amplified with the primers containing Bpu1102 I restriction site. The obtained PCR product was digested with the Bpu1102I and ligated in pET 27 bZBG13.1.1 predigested with Bpu1102I. The ligated DNA was further transformed into competent E. coli Top10F′ cells and the transformation mix was plated on Luria agar plates containing kanamycin. The positive clones were identified on the basis of their utilizing kanamycin resistance for growth on the above Petri plates and further restriction digestion of the plasmid DNA derived from them. The restriction enzymes, such as SspI and BamHI, which is supposed to digest both the vector and the gene insert obtained from such clones. One such clone giving desired fragment lengths of digested plasmid DNA samples was selected as a positive clone. This clone was named pZRC2G-1ZBG2.0.1c1. Plasmid DNA isolated from this clone was transformed into the E. coli expression host, BL21 (DE3), and plated on kanamycin containing Luria Agar plates followed by incubation at 37° C. for overnight. Colonies picked from this plate were grown in Luria Broth containing kanamycin followed by induction with suitable concentration 2 mM Make specific of IPTG for expression analysis. Simultaneously the plasmid DNA isolated from these cultures was further subjected to restriction digestion analysis using restriction enzymes SspI to confirm the correctness of the clone. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of this clone known as pZRC2G-1ZBG2.0.1c1 BL21(DE3) was used for the desired enzymatic activity in preparation of glycerol stocks. This clone pZRC2G-1ZBG2.0.1c1 BL21(DE3) was used as a source of enzymatic polypeptide of Seq ID no 1 and Seq ID no. 2 for subsequent biocatalysis studies.

### Example 4

### Construction of Plasmid pZRC2G-1ZBG2.0.1c2 for Coexpression of Oxidoreductase and Cofactor Regenerating Enzyme

The plasmid pET27bZBG2.0.1 prepared according to example 1 containing a oxidoreductase gene deduced from the polypeptide sequence as shown in sequence id no. 1 was used for the co-expression of GDH derived from DNA sequence id no. 1 in single expression system.

A DNA sequence deduced from the above polypeptide sequence as shown in sequence id no. 1 optimized for expression in E. coli and cloned in a pET27 b plasmid vector i.e. pET 27 b ZBG 2.0.1 was used for the cloning and expression of another expression cassette of DNA sequence id no. 2 deduced from the cloned vector pET 27 b ZBG 13.1.1 in a duet manner wherein the both poly peptides are expressed in a single host system. The expressions construct of the pET 11a ZBG13.1.1 containing T7 promoter RBS and the ZBG 13.1.1 gene was amplified with the primers containing Bpu1102 I restriction site. The obtained PCR product was digested with the Bpu1102I and ligated in pET 27bZBG2.0.1 predigested with Bpu1102I. The ligated DNA was further transformed into competent E. coli Top10F′ cells and the transformation mix was plated on Luria agar plates containing kanamycin. The positive clones were identified on the basis of their utilizing kanamycin resistance for growth on the above Petri plates and further restriction digestion of the plasmid DNA derived from them. The restriction enzymes, such as SspI, which is supposed to digest both the vector and the gene insert obtained from such clones was used for screening. One such clone giving desired fragment lengths of digested plasmid DNA samples was selected as a positive clone. This clone was named pZRC2G-1ZBG2.0.1c2. Plasmid DNA isolated from this clone was transformed into the E. coli expression host, BL21 (DE3), and plated on kanamycin containing Luria Agar plates followed by incubation at 37° C. for overnight. Colonies picked from this plate were grown in Luria Broth containing kanamycin followed by induction with suitable concentration 2 mM of IPTG for expression analysis. Simultaneously the plasmid DNA isolated from these uninduced cultures was further subjected to restriction digestion analysis using restriction enzymes SspI to confirm the correctness of the clone. IPTG induced cultures were lysed and clarified lysates obtained after centrifugation were subjected to SDS-PAGE analysis to confirm induced expression of polypeptide of correct size. After confirming the restriction fragment analysis and expression analysis, the fresh culture of this clone known as pZRC2G-1ZBG2.0.1c2 BL21(DE3) was used for the preparation of glycerol stocks. This clone pZRC2G-1ZBG2.0.1c2 BL21(DE3) was used as a source of enzymatic polypeptide of Seq ID no 1 and Seq ID No. 2 for subsequent biocatalysis studies.

### Example 5

### Enzyme Preparation of Oxidoreductase Obtained from pET27bZBG2.0.1 and Glucose Dehyrogenase Obtained from pET27bZBG13.1.1 at Shake Flask Level

The recombinant/transformed E. coli obtained from example 1 and 2 containing pET27bZBG2.0.1 and pET27bZBG13.1.1 respectively, were separately cultured in 50 ml Luria Bertani (LB) medium, containing 10 g peptone, 5 g yeast extract, 10 g NaCl per liter of water with 75 μg/ml Kanamycin and cultivated for at least 16 h at 37° C. with shaking at 200 rpm. Activated culture further inoculated to 750 ml LB medium containing 75 μg/ml kanamycin to set the optical density at 600 nm (OD₆₀₀). Expression of protein was induced with 1 mM iso-propyl β-D-thiogalactoside (IPTG), when culture OD₆₀₀ was 0.6 to 0.8 and shaken at 200 rpm at 37° C. for at least 16 h. Cells were harvested by centrifugation for 15 min at 7000 rpm at 4° C. and supernatant discarded.

The cell pellet was re-suspended in cold 100 mM Potassium Phosphate Buffer (pH 7.0) (KPB) and harvested as mentioned above. Washed cells were re-suspended in 10 volumes of cold 100 mM KPB (pH 7.0) containing 1 mg/ml lysozyme, 1 mm PMSF and 1 mM EDTA and homogenous suspension subjected to cell lysis by ultrasonic processor (Sonics), while maintained temperature at 4° C. Cell debris was removed by centrifugation for 60 min at 12000 rpm at 4° C. The clear crude lysate supernatant (cell free extract) was lyophilized and lyophilized powder stored at 4° C. for further use.

### Example 6

### Enzymatic Activity of Oxidoreductase Obtained from pET27bZBG2.0.1 and Glucose Dehydrogenase Obtained from pET27bZBG13.1.1

The ketoreductase activity of clear crude lysate of pET27bZBG2.0.1 obtained in example 5 was assayed spectrophotometrically in an NADPH depended assay at 340 nm (OD₃₄₀) at 25° C. The 1.0 ml standard assay mixture comprised of 100 mM KPB (pH 7.0), 0.1 mM NADPH, and 2.5 mM 3-quinuclidinone. The reaction was initiated by addition of 100 μl of crude lysate of pET27bZBG2.0.1 and monitored up to 10 min. The 1 Unit (U) of enzyme was defined as amount of enzyme required to generate 1 μmole of NADPH in 1 min. The ketoreductase activity of pET27bZBG2.0.1 showed 0.2 U/ml of cell free extract.

The glucose dehydrogenase (GDH) activity of clear crude lysate of pET27bZBG13.1.1 obtained in example 5 was assayed spectrophotometrically in an NADPH depended assay at 340 nm (OD₃₄₀) at 25° C. The 1.0 ml standard assay mixture comprised of 100 mM KPB (pH 7.8), 2 mM NADP and 0.1M Glucose. The reaction was initiated by addition of 1000 with suitable dilution of crude lysate of pET27bZBG13.1.1 and monitored up to 10 min. The 1 Unit (U) of enzyme was defined as amount of enzyme required to oxidized 1 μmole of NADPH in 1 min. The glucose dehydrogenase activity of pET27bZBG13.1.1 showed 28 U/ml of cell free extract.

### Example 7

### Synthesis of (R)-3-Quinuclidinol from 3-Quinuclidinone Using Oxidoreductase Obtained from pET27bZBG2.0.1 by Substrate Coupled Cofactor Regeneration System

Crude lyophilized powder obtained from 4 gm of harvested cells as mentioned in example 5 was used to charged the reaction, which comprised of 100 mg (0.8 mmoles) of 3-Quinuclidinone, 1.27 mM of Nicotinamide adenine dinucleotide phosphate disodium salt (NADP⁺), 10% (v/v) of 2-propanol and 0.1M potassium phosphate buffer (pH 7.0). The homogenous reaction preparation was incubated at 37° C.±0.5° C. under shaking conditions, 200 rpm. After 48 h, the reaction mixture was alkalified by addition of saturated K₂CO₃ solution and extracted with equal volume of ethyl acetate. The upper organic layer was further analyzed by gas chromatography (GC) analysis in fused silica capillary column, BP-5 (30m×0.32 mm ID, 0.25 g or equivalent). The column temperature was 220° C. and detection temperature was 250° C. The retention time of each compound was around 5.8 min for 3-quinuclidinone and around 6.2 min for 3-quinuclidinol in FID detector (Flame Ionized Detector). The purity of formed-3-quinuclidinol was analyzed by gas chromatography by using HP-5 (30m×0.32 mm ID, 0.25μ or equivalent). The column temperature was 250° C. and detection temperature was 280° C. The retention time of the compound was around 9.6 min in FID detector (Flame Ionized Detector). The optical purity of the (R)-3-quinuclidinol was determined by GC analysis by using Gamma DEX-TM-225 capillary column (30m×0.25 mm ID, 0.25μ or equivalent). The retention time of (S) isomer is around 3.8 and for (R) around 4.1. Samples analyzed by mentioned method, showed a 99.47% GC purity and >95% ee of (R)-3-Quinuclidinol.

### Example 8

### Synthesis of (R)-3-Quinuclidinol from 3-Quinuclidinone Using Oxidoreductase Obtained from pET27bZBG2.0.1 by Enzyme Coupled Cofactor Regeneration System

The reaction mixture consist of 100 mg (0.8 mmoles) 3-Quinuclidinone, 1.27 mM NADP⁺ and 0.694 moles of glucose dissolved in 0.1 M Potassium phosphate buffer (pH 7.0) was initiated by adding crude lyophilized powder of each enzyme pET27bZBG2.0.1 derived ketoreductase and pET27bZBG 13.1.1 derived glucose dehydrogenase, obtained from harvested cells as mentioned in Example 5. The homogenous reaction preparation was incubated at 37°±0.5 C under shaking conditions, 200 rpm. After 3 h the reaction mixture was alkalified by addition of saturated K₂CO₃ solution and extracted with equal volume of ethyl acetate. The upper organic layer was further analyzed by gas chromatography (GC) analysis as mentioned in Example 7 which showed a >99.56% GC purity a >95% ee of (R)-3-Quinuclidinol.

### Example 9

### Synthesis of (R)-3-Quinuclidinol from 3-Quinuclidinone Using Oxidoreductase Obtained from pET27bZBG2.0.1 by Enzyme Coupled Cofactor Regeneration System

30 g (0.24 moles) of 3-Quinuclidinone added as free base to 150 ml of water containing 3.75 gm of crude lyophilized powder of pET27bZBG13.1.1 obtained in example 5 and 0.694 moles of glucose. The reaction is initiated by adding 7.5 gm crude lyophilized powder of pET27bZBG2.0.1 obtained in example 5 to the reaction mixture. The homogenous reaction preparation was incubated at 37° C.±2.0 under shaking conditions.

After 12 hrs the reaction mixture was alkalified with NaOH and extracted in equal volumes of n-Butanol. Upon evaporating the solvent the desired product was obtained in not less than 85% yield.

The product was further analyzed by GC analysis followed by chiral GC analysis, which showed a >99% GC purity and >95% ee of (R)-3-Quinuclidinol.

### Example 10

### Preparation of Co-Expressed Enzyme Obtained from pZRC2G-1ZBG2.0.1c1 at Fermentor Level

Fermentation was carried out in agitated and aerated 30 L fermentor with 10 L of growth medium containing; Glucose 10 g/L, Citric acid 1.7 g/L, Yeast extract 10 g/L, Di-potassium hydrogen phosphate 4 g/L, Magnesium sulfate heptahydrate 1.2 g/L, Trace metal solution 20 ml/L (comprised: 0.162 g/L Ferrous chloride hexahydrate, 0.0094 g/L Zinc chloride, 0.12 g/L Cobalt chloride, 0.012 g/L sodium molybdate dihydrate, 2.40 g/L copper chloride, 0.5 g/L Boric acid) and kanamycin monosulfate 75 mg/L. The recombinant pZRC2G-1ZBG2.0.1c1 grown LB in shake flask as mentioned in example 5 with late exponential cultures was used to inoculate fermentor to set 0.5 OD₆₀₀. The aeration was maintained at 50-70% saturation with 5-15 L/min of dissolved oxygen and agitated at 200-1000 rpm. The pH of the culture was maintained at 6.8±0.2 with 12.5% (v/v) ammonium hydroxide solution. Growth of the culture was maintained with a feed solution of growth medium containing; Glucose 700 g/L, Yeast extract 50 g/L, Trace metal 20 ml/L, Magnesium sulfate heptahydrate 10 g/L, kanamycin monosulfate 750 mg/L. Expression of protein was induced with iso-propyl β-D-thiogalactoside (IPTG) at the final concentration of 0.06 mM/g of DCW (Dry cell weight), when culture OD₆₀₀ reaches around 50.0±2.0. The fermentation continued further for another 12±2 hrs with feed solution of production medium containing Glucose 200 g/L, Yeast extract 200 g/L and kanamycin monosulfate 750 mg/L. The culture was chilled to 15° C.±5.0 and broth harvested by centrifugation 6500 rpm for 20 min at 4° C. Cell pellet collected after washing with 0.05M potassium phosphate buffer (pH 7.0) by centrifugation at 8000 rpm for 20 min. Cells were stored at 4° C. or preserved at −70° C. until used further for the mentioned biocatalytic conversion.

The enzymatic activity of oxidoreductase and glucose dehydrogenase in co-expressed pZRC2G-1ZBG2.0.1c1 was assayed spectrophotometrically in NADPH depended assay at 340 nm (OD₃₄₀) at 25° C. as mentioned in example 6. The enzyme activity of 1 ml of cell free extract derived pZRC2G-1ZBG2.0.1c1 showed 0.214 U and 34U for ketoreductase and glucose dehydrogenase, respectively.

### Example 11

### Synthesis of (R)-3-Quinuclidinol from 3-Quinuclidinone Using Whole-Cell Catalyst co-expressing oxidoreductase and glucose dehydrogenase

10 gm (0.08 moles) of 3-Quinuclidinone was added to 50 ml of water containing Glucose (0.12 moles). The reaction is initiated by adding 15 gm whole cells prepared as mentioned in the example 10 The homogeneous reaction preparation was incubated at room temp under shaking condition for 4-5 hours. The reaction mixture was alkalified with NaOH and extracted in equal volumes of n-Butanol. Upon evaporating the solvent the desired product was obtained in not less than 85% yield. The product was future analyzed by GC analysis followed by chiral GC analysis. Which showed >99.70% GC purity and >95% ee of (R)-3-Quinuclidinol.

### Example 12

### Synthesis of (R)-3-Quinuclidinol from 3-Quinuclidinone Using Coexpressed Lyophilized Oxidoreductase and Glucose Dehydrogenase

The whole cell pellet as prepared in example 10 was suspended in the 10 volumes of pre-chilled 0.05M potassium phosphate buffer (pH 7.0) in chilled condition. The homogenous single cell preparation subjected to cell disruption by passing though high pressure homogenizer at 1000±100 psig at 4° C., in subsequent two cycles. The resulting homogenate clarified by centrifugation at 8000 rpm for 120 min. The clear supernatant collected and subjected to lyophilization. The crude lyophilized powder used further as mentioned in below biocatalytic conversion. 50 gm (0.4 moles) of 3-Quinuclidine was added to 250 ml of water containing glucose (0.6 moles) and 50 mg of NADP and the reaction was initiated by adding 15 gm crude lyophilized powder. The homogeneous reaction preparation was incubated at room temp under shaking condition for 6-7 hours. The reaction mixture was alkalified with NaOH and extracted in equal volumes of n-Butanol. Upon evaporating the solvent the desired product was obtained in not, less than 85% yield. The product was further analyzed by GC analysis followed by chiral GC analysis. Which showed >99.67% GC purity & >95% ee of (R)-3-Quinuclidinol.

1. A process for preparing the 3-quinuclidinol of formula (I) 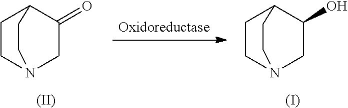
which comprises
(a) reacting the quinuclidinone of formula (II) with an oxidoreductase enzyme of SEQ ID NO:1 or its suitable variants in the presence of suitable cofactor in suitable solvent; and
(b) isolating the 3-quinuclidinol wherein the optical purity is at least 95%.

2. The process as claimed in claim 1, wherein the reaction is carried out at suitable pH.

3. The process as claimed in claim 1, wherein the oxidoreductase enzyme is a ketoreductase.

4. The process as claimed in claim 1, wherein the oxidoreductase enzyme is a short chain alcohol dehydrogenase.

5. The process as claimed in claim 1, wherein cofactor is continuously regenerated through enzyme based regeneration system wherein the enzyme oxidizes the suitable co-substrate to regenerate co-factor.

6. The process as claimed in claim 1 wherein the enzyme employed in co-factor regeneration is selected from glucose dehydrogenase, formate dehydrogenase, malate dehydrogenase, glucose-6-phosphate dehydrogenase, phosphite dehydrogenase.

7. The process as claimed in claim 6, wherein the enzyme employed in co-factor regeneration is glucose dehydrogenase as set forth in SEQ ID NO:2.

8. The process as claimed in claim 1, wherein cofactor is continuously regenerated through substrate based co-factor regeneration system wherein the enzyme oxidize the suitable co-substrate to regenerate co-factor.

9. The process as claimed in claim 8, wherein the enzyme is an oxidoreductase.

10. The process as claimed in claim 8, wherein the co-substrate is selected from n-butanol, Iso propyl alcohol, ethyl acetate, butyl acetate, toluene, chloroform, n-hexane, ethanol, acetone, dimethyl sulfoxide, and acetonitrile.

11. The process as claimed in claim 10, wherein the co-substrate is iso propyl alcohol.

12. The process as claimed in claim 1, wherein the suitable solvent is selected from water, iso-propyl alcohol, dimethyl sulfoxide or their suitable mixtures.

13. The process as claimed in claim 1, wherein the oxidoreductase enzyme of SEQ ID NO:1 is cloned in a vector and subsequently expressed in a suitable recombinant whole cell.

14. The process as claimed in claim 13 wherein the recombinant whole cell further co-express polypeptide having potential to regenerate cofactor from oxidized NAD(P).

15. The process as claimed in claim 13, wherein the expression vector comprises
a) at least one region that control the replication and maintenance of said vector in the host cell;
b) first promoter operably linked to the nucleotide sequence setforth in SEQ ID NO:1 (ZBG2.0.1) encoding the oxidoreductase enzyme;
c) second promoter operably linked to the nucleotide sequence SEQ ID NO:2 (ZBG13.1.1) encoding polypeptide having potential to regenerate co-factor; and
d) suitable antibiotic marker.

16. The process as claimed in claim 13, wherein the recombinant whole cell comprising an oxidoreductase enzyme and a glucose dehydrogenase has accession number MTCC 5621.

17. A process for the production of the compound of formula (I) as claimed in claim 1, which comprises
a) reacting the compound of formula (II) 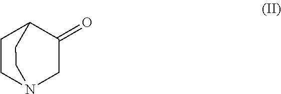
with suitable recombinant whole cell having accession number MTCC 5621 which comprises an expression vector which co-expresses the oxidoreductase enzyme of SEQ ID NO:1 or its variants and polypeptide having potential to regenerate co-factor in suitable solvents;
b) maintaining the pH during the reaction, as required; and
c) isolating of the compound of formula (I).

18. The process as claimed in claim 8, wherein the concentration of substrate is selected from 0.1 to 30% w/v.

19. The process as claimed in claim 1, wherein the oxidoreductase enzyme is isolated from species of Saccharomyces, preferably Saccharomyces cerevisiae.

20. The process as claimed in claim 1, wherein the glucose dehydrogenase is isolated from Bacillus megaterium.

21. The process as claimed in claim 1, wherein the cofactor is NAD(P)H and NAD(P).

22. The process as claimed in claim 2, wherein the pH is maintained at 5.5 to 8.5, preferably 6.5 to 8 and most preferably 7 to 7.5

23. A vector for the expression of chiral alcohol which comprises:
a) at least one region that control the replication;
b) suitable promoter operably linked to the desired polynucleotide sequence of SEQ ID NO:1 or its variants; and
c) an antibiotic marker.

24. The vector as claimed in claim 23, which further comprises the polynucleotide sequence of SEQ ID NO:2 or its variants.

25. The vector as claimed in claim 23, which expresses the oxidoreductase enzyme is pET27bZBG2.0.1.

26. The vector as claimed in claim 23, which expresses the Glucose dehydrogenase enzyme is pET27bZBG 13.1.1

27. The vector as claimed in claim 23 co-expresses the oxidoreductase and Glucose dehydrogenase enzymes is pZRC2G-IZBG2.0.1c1.

28. The vector as claimed in claim 23 co-expresses the oxidoreductase and Glucose dehydrogenase enzymes is pZRC2G-IZBG2.0.1c2

29. The process as claimed in claim 1, further comprising a buffer.

30. The process as claimed in claim 29, wherein the buffer is selected from sodium succinate, sodium citrate and sodium phosphate.
